(19) United States
(12) Patent Application Publication
(10) Pub. No.: US 2004/0176668 A1
(43) Pub. Date: Sep. 9, 2004

(54) Support and sensing apparatus

(76) Inventor: James A. Goldstein, Bloomfield Hills, MI (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/807,745

(22) Filed: Mar. 24, 2004

Related U.S. Application Data
(63) Continuation-in-part of application No. 10/721,032, filed on Nov. 24, 2003.
Application No. 10/721,032 is a division of application No. 09/990,073, filed on Nov. 21, 2001, now Pat. No. 6,653,648.
Application No. 09/990,073 is a continuation-in-part of application No. 09/638,772, filed on Aug. 15, 2000, now Pat. No. 6,448,571.

(51) Int. Cl. A61B 5/00; A47B 7/00

(52) U.S. Cl. 600/300; 5/621; 5/623; 600/323; 600/549; 600/502; 600/509; 600/485

(57) ABSTRACT

A support apparatus for supporting at least a portion of a patient's limb during a procedure. The apparatus includes a support having a surface for receiving at least a portion of the patient's limb thereon to support the limb during the procedure, and a sensor mounted on the support such that the sensor is at least partially attached to the support without an adhesive. The sensor is adapted to communicate with the patient's limb for measuring a physiological parameter of the patient on the limb when the limb is supported by the support.

### RELATED APPLICATION DATA

This application is a continuation-in-part of Ser. No. 10/721,032, filed Nov. 24, 2003, which is a divisional of Ser. No. 09/990,073, filed Nov. 21, 2001, now U.S. Pat. No. 6,653,648, which is a continuation-in-part of Ser. No. 09/638,772, filed Aug. 15, 2000, now U.S. Pat. No. 6,448,571, all of which are incorporated herein by reference to the extent permitted by law.

### BACKGROUND OF THE INVENTION

The present invention relates generally to medical and surgical procedures, and more specifically to an apparatus for supporting and restraining a patient's limb during a medical and/or surgical procedure.

During many medical and/or surgical procedures patients are often restrained to facilitate completion of the procedure with as little discomfort and/or injury to the patient as possible. Restraining the patient may further prevent damage to the equipment used in the procedure. It may also be necessary to monitor the patient during some procedures to ensure the safety of the patient and facilitate completing the procedure. For example, physiological parameters such as temperature, pulse, blood pressure, etc. may be monitored to verify they are acceptable for the particular procedure being performed on the patient. However, restraining the arms and legs of the patient may interfere with monitoring these parameters of the patient.

### SUMMARY OF THE INVENTION

In one aspect, the present invention includes a support apparatus for supporting at least a portion of a patient's limb during a procedure. The support apparatus includes a support having a surface for receiving at least a portion of the patient's limb thereon to support the limb during the procedure, and a sensor mounted on the support such that the sensor is at least partially attached to the support without an adhesive. The sensor is adapted to communicate with the patient's limb for measuring a physiological parameter of the patient on the limb when the limb is supported by the support.

In another aspect, the present invention includes a support apparatus for restraining movement of at least a portion of a patient's limb during a procedure. The apparatus includes a support for receiving at least a portion of the patient's limb thereon to support the limb during the procedure, and a restraint mounted on the support for engaging the limb to restrain movement of the limb. The restraint includes a sensor adapted to communicate with the patient's limb for measuring a physiological parameter of the patient on the limb when the limb is received by the restraint.

In another aspect, the present invention includes a support apparatus for restraining movement of at least a portion of a patient's limb during a procedure. The apparatus includes a support for receiving at least a portion of the patient's limb thereon to support the limb during the procedure, and a glove mounted on the support and adapted to receive at least a portion of a hand of the patient's limb. The glove has a first portion adapted to receive at least a portion of a first digit of the hand, and a second portion separate from the first portion and adapted to receive at least a portion of a second digit of the hand.

In yet another aspect, the present invention includes a glove assembly for measuring a physiological parameter of patient during a procedure. The glove assembly includes a glove adapted to receive at least a portion a hand of the patient, and a sleeve extending from the glove and adapted to receive at least a portion of an arm of the patient. The sleeve is adapted to extend beyond a wrist of the patient and along at least a portion of the arm when the arm is received by the sleeve. The glove assembly also includes a sensor mounted on at least one of the glove and the sleeve. The sensor is adapted to communicate with the patient for measuring the physiological parameter when the arm is received by the sleeve.

In even another aspect, the present invention includes a support apparatus for supporting at least a portion of a patient's limb during a procedure. The apparatus includes a support having a surface for receiving at least a portion of the patient's limb thereon to support the limb during the procedure, and a sensor mounted on the support and including a measuring region facing away from the support surface for engaging the patient's limb when received by the support for measuring a physiological parameter of the patient.

Other features of the present invention will be in part apparent and in part pointed out hereinafter.

### BRIEF DESCRIPTION OF THE DRAWINGS

FIG. 1 is a top plan view of a support apparatus of a first embodiment of the present invention;

FIG. 2 is a perspective of a support apparatus of a second embodiment of the present invention;

FIG. 3 is a perspective of a support apparatus similar to the second embodiment and having a surface contoured to generally correspond to a patient's arm and hand;

FIG. 4 is a perspective of a support apparatus similar to the second embodiment including a restraint;

FIG. 5 is a perspective of a support apparatus similar to the second embodiment including a glove;

FIG. 6 is a perspective of a portion of a glove of the present invention;

FIG. 7 is a perspective of a support apparatus of a third embodiment of the present invention; and

FIG. 8 is a perspective of a glove assembly of the present invention.

Corresponding reference characters indicate corresponding parts throughout the several views of the drawings.

### DETAILED DESCRIPTION OF THE PREFERRED EMBODIMENT

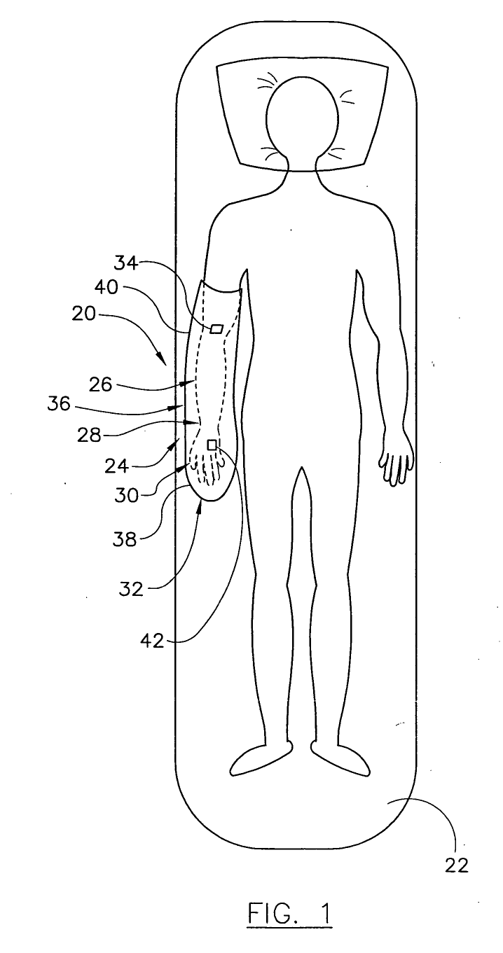

Referring now to the drawings, and more specifically to FIG. 1, one embodiment of a support apparatus of the present invention is designated in its entirety by the reference numeral 20. The apparatus 20 includes a table 22 having a support (generally designated by 24) for supporting a patient's limb (e.g., the arm, wrist, and hand generally designated by 26, 28, 30, respectively) during a procedure. The apparatus 20 also includes a restraint (generally designated by 32) mounted on the support 24 for restraining movement of at least a portion of the limb such as the wrist 28, the hand 30, and a portion of the arm 26. The restraint 32 includes a sensor 34 for measuring a physiological parameter (e.g., a pulse) of the patient. The sensor 34 is positioned to measure the parameter at a predetermined location on the patient's limb such as the patient's arm 26. As described below, by supporting and restraining movement of a patient's limb (or a portion thereof) and/or measuring a physiological parameter on the limb, the apparatus of the present invention may facilitate completion of medical and/or surgical procedure(s), including measurement of the parameter, with as little discomfort and/or injury to the patient, and damage to the various components of the apparatus and/or other equipment (not shown). Furthermore, the apparatus may facilitate quick and convenient measurement of one or more of the same and/or different physiological parameter(s) of the patient during the procedure(s).

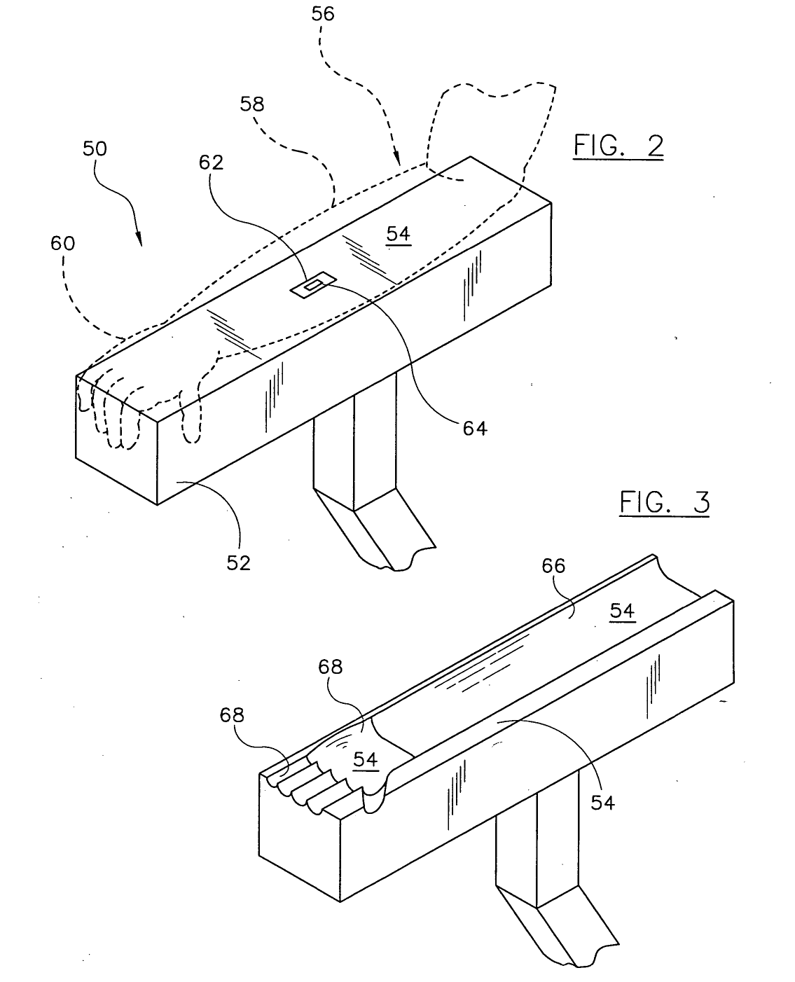

Although other physiological parameters may be measured without departing from the scope of the present invention, in one embodiment the physiological parameter measured by the sensor is an electrocardiogram, a temperature, a pulse, a blood pressure, or an oxygen saturation. Accordingly, the sensor may be a temperature sensor, a pulse sensor, a blood pressure sensor, an electrocardiograph, or an oxygen saturation sensor. The support apparatus of the present invention may include any number of sensors for measuring the same and/or different particular physiological parameters of the patient on various portions of the patient's limb. Moreover, the sensors may be mounted anywhere on the supports and/or the restraints of the present invention so they can communicate with the patient's limb for measuring the physiological parameter(s) at a desired location on the limb. For example, as shown in FIG. 1 the restraint 32 includes a glove assembly (generally designated by 36) having a glove 38 sized and shaped to receive the hand 30 and a sleeve 40 extending from the glove adapted to receive the wrist 28 and a lower portion of the arm 26. The sleeve 40 includes sensor 34 and the glove 38 includes sensor 42 mounted thereon for measuring a physiological parameter of the patient on the arm 26 and the hand 30, respectively. Another example includes mounting a sensor on a support of the present invention, as shown in FIG. 2.

Although a support apparatus of the present invention may be incorporated in a table or other device for supporting more than a patient's limb without departing from the scope of the present invention, the support apparatus may be configured to support only a patient's limb, or only a portion thereof. For example, as shown in FIG. 2 a support apparatus of another embodiment (generally designated by 50) includes a support 52 having a surface 54 for receiving a portion of a patient's limb (generally designated by 56) thereon to support the limb during a procedure. In this embodiment, the supported limb includes an arm 58 and a hand 60. The support 52 also includes a sensor 62 for measuring a physiological parameter of the patient as described herein. The sensor 62 may be mounted on the support 52 in any suitable manner (e.g., threaded fasteners, adhesive, and/or hook and loop fasteners). In one embodiment, the sensor 62 is mounted on the support in a manner such that the sensor is at least partially attached to the support 52 without an adhesive (i.e., if the sensor is mounted on the support with an adhesive the adhesive is not the only attachment between the sensor and the support).

The sensor 62 is mounted on the support 52 in a manner such that the sensor is adapted to communicate with the patient's limb when the limb is supported by the support for measuring the physiological parameter(s) of the patient. Although other configurations may be used without departing from the scope of the present invention (e.g., a sensor (not shown) mounted on the support 52 such that the sensor communicates with a portion of the limb 56 not in contact with the surface 54), the sensor 62 is adapted to communicate with a portion of the limb in contact with the surface 54, as shown in FIG. 2. More specifically, the sensor 62 includes a measuring region 64 facing away from the support 52 for engaging the portion of the limb 56 in contact with the surface 54 when the limb is received by the support. As discussed above, the support 52 may include any number of sensors each located anywhere on the support suitable for measuring the parameter(s). Although the surface 54 of the support 52 may have any suitable shape (e.g., a flat surface), the surface 54, or a portion thereof, may have a contour generally corresponding to one or more portions of the limb 56. For example, as shown in FIG. 3 a portion of the surface 54 has a contour 66 generally corresponding to a portion of the arm 58 (FIG. 2), and a portion of the surface 54 has a contour 68 generally corresponding to a portion of the hand 60 (FIG. 2). The contour of the surface 54 may be any suitable size and shape generally corresponding to a portion of the limb 56.

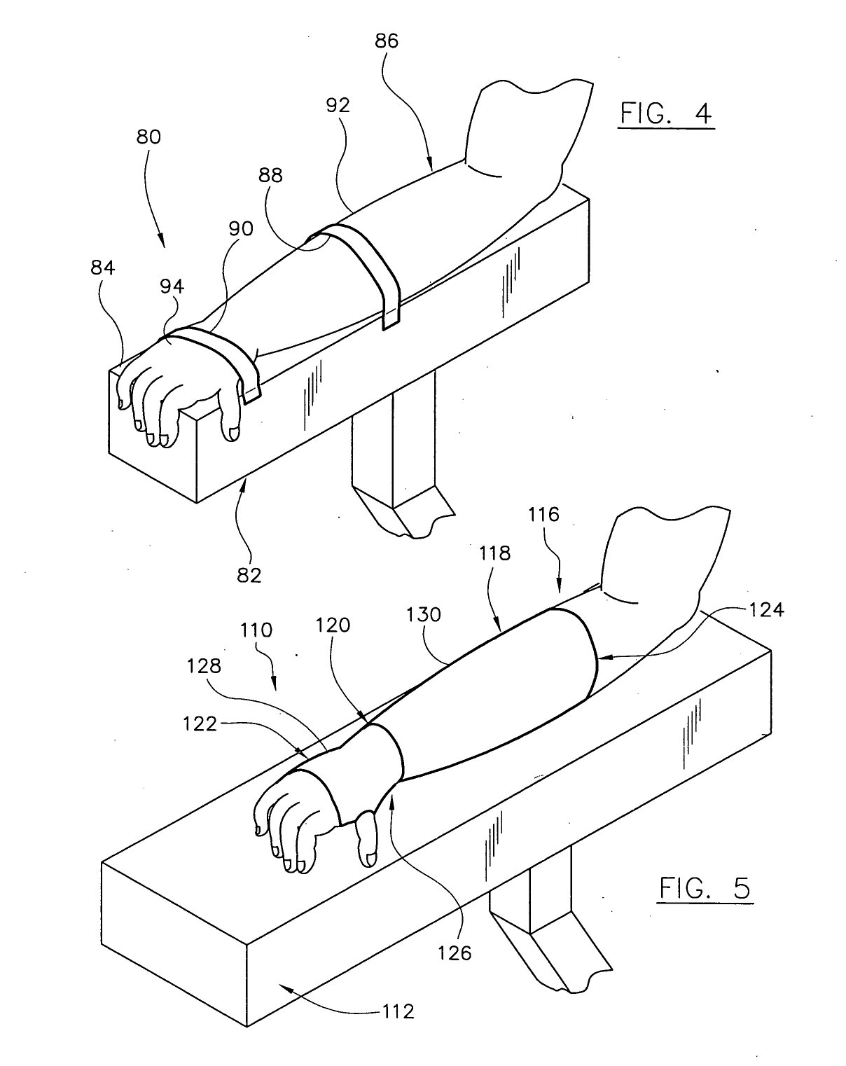

Although the support apparatus of the present invention may not include a restraint, a support apparatus of one embodiment (generally designated by 80 in FIG. 4) includes a support (generally designated by 82) having a plurality of restraints 88, 90. The support 82 has a surface 84 for receiving a portion of a patient's limb (generally designated by 86) thereon to support the limb during a procedure. The plurality of restraints 88, 90 are mounted on the support 82 for engaging the limb 86 to restrain movement of the limb during the procedure(s). The limb includes an arm 92 and a hand 94.

Although the restraints of the present invention are specifically described and illustrated herein, the present invention may generally include any number of restraints, each of which may be any type and/or configuration (e.g., size, shape, location, and/or arrangement) suitable for restraining movement of the patient's limb (or a portion thereof) during the procedure(s), and/or suitable for measuring a physiological parameter on the limb during the procedure(s) when the restraint includes a sensor. As shown in FIG. 4, in one embodiment the restraints 88, 90 are straps adapted to receive a portion of the arm 92 and the hand 94 for restraining movement of the arm and the hand, and for measuring the physiological parameter(s) on the arm and/or hand, respectively, when one or both of the restraints 88, 90 includes a sensor. Another example of a restraint within the scope of the present invention is the restraint 32 (FIG. 1) including the glove 38 (FIG. 1) and the sleeve 40 (FIG. 1).

Another example of a restraint of the present invention is illustrated in FIG. 5. More specifically, a support apparatus (generally designated by 110) includes a support (generally designated by 112) for supporting a patient's limb (generally designated by 116) during a procedure. The limb 116 includes an arm (generally designated by 118), a wrist (generally designated by 120), and a hand (generally designated by 122). The apparatus 110 also includes a restraint (generally designated by 124) mounted on the support 112 for restraining movement of the wrist 120, the hand 122, and a portion of the arm 118 during the procedure. The restraint 124 includes a glove assembly (generally designated by 126) including a glove 128 adapted to receive a portion of the hand 122 for restraining movement of the hand. Further, the glove 128 may by adapted for measuring a physiological parameter on the hand when the glove includes a sensor. The glove assembly 126 also includes a sleeve 130 extending from the glove 128. The sleeve 130 is adapted to receive a portion of the arm 118 for restraining movement of the arm and for measuring a physiological parameter on the arm when the sleeve includes a sensor. As shown in FIG. 5, in one embodiment the sleeve 130 is adapted to extend beyond the wrist 120 and along at least a lower portion of the arm 118 when the arm is received by the sleeve.

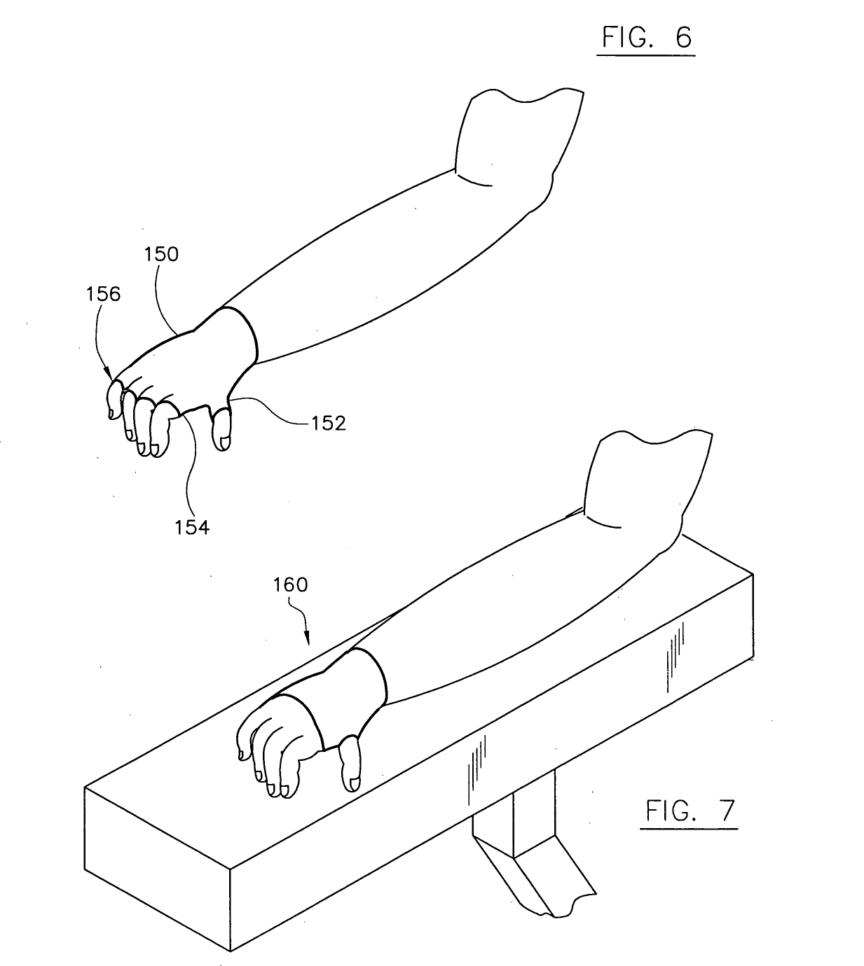

Although the gloves and glove assemblies of the present invention are specifically described and illustrated herein, the present invention may generally include gloves and glove assemblies of any type and/or configuration (e.g., size, shape, location, and/or arrangement) suitable for restraining movement of the patient's limb (or a portion thereof) during the procedure(s), and/or suitable for measuring a physiological parameter on the limb during the procedure(s) when the gloves and/or assemblies include a sensor. For example, the glove assembly of the present invention may be the glove assembly 126 shown in FIG. 5, wherein the assembly includes a glove 128 adapted to receive a portion of a hand. Another example is the glove assembly 36 shown in FIG. 1 wherein the glove 38 generally receives the entire hand 30 without separating digits of the hand. Furthermore, as illustrated in FIG. 6 a glove 150 of the present invention may include at least two portions 152, 154, each for separately receiving a digit (e.g., a finger or a thumb) of a hand (generally designated by 156). The portions 152, 154 may extend to completely surround the entire digits, or alternatively may partially surround the digits as shown in FIG. 6. Moreover, as illustrated in FIG. 7 a glove assembly 160 of the present invention may not include a sleeve.

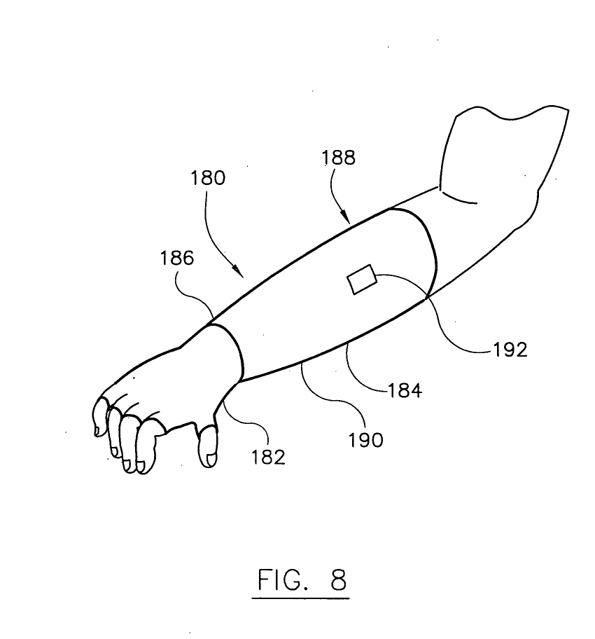

As illustrated in FIG. 8, a glove assembly (generally designated by 180) of the present invention may be used independently from a support to measure a physiological parameter of a patient during a medical and/or surgical procedure. More specifically the glove assembly 180 includes a glove 182 and a sleeve 184 extending from the glove. The sleeve 184 is adapted to extend beyond a wrist 186 of a limb (generally designated by 188) of the patient and along at least a portion of an arm 190 of the limb when the arm is received by the sleeve. The assembly 180 also includes a sensor 192 mounted thereon and adapted to communicate with the limb for measuring the physiological parameter(s). Although the sensor 192 may be mounted anywhere on the glove assembly 180, in one embodiment shown in FIG. 8 the sensor 192 is mounted on the sleeve 184 and adapted to communicate with the arm 190 for measuring the parameter(s) on the arm.

The above-described apparatus and assemblies are cost-effective and reliable for restraining movement of a patient's limb, or a portion thereof, and measuring a physiological parameter of the patient during a procedure. Specifically, by supporting and restraining movement of a patient's limb, or a portion thereof, the apparatus and assemblies of the present invention may facilitate completion of procedure(s) with as little discomfort and/or injury to the patient as possible. Furthermore, restraining movement of the limb, or a portion thereof, may prevent damage to the various components of the assemblies, apparatus, and/or other equipment used in the procedure(s). The apparatus and assemblies of the present invention may also facilitate quick and convenient measurement of one or more of the same and/or different physiological parameter(s) of a patient during the procedure(s). Moreover, when measurement of the physiological parameter(s) is combined with supporting and/or restraining the limb, the apparatus and assemblies may facilitate measuring the physiological parameter with as little discomfort and/or injury to the patient, disruption of the procedure(s), and/or damage to the various components of the apparatus, assemblies and/or other equipment used in the procedure(s) as possible.

Although the invention is herein described and illustrated in association with a human limb, and more specifically in association with restraining and/or measuring a physiological parameter of a human arm, hand, and/or wrist, it should be understood that the present invention is generally applicable to restraining and/or measuring a physiological parameter of any limb of a human or other animal. Accordingly, practice of the present invention is not limited to restraining and/or measuring a physiological parameter of a human arm and/or hand.

Exemplary embodiments of support apparatus and glove assemblies are described above in detail. The apparatus and assemblies are not limited to the specific embodiments described herein, but rather, components of each apparatus and assembly may be utilized independently and separately from other components described herein. Each support apparatus and glove assembly component can also be used in combination with other support apparatus and/or glove assembly components.

As used herein, the term “patient” is intended to mean any animal.

When introducing elements of the present invention or the preferred embodiment(s) thereof, the articles “a”, “an”, “the” and “said” are intended to mean that there are one or more of the elements. The term “plurality” is intended to mean there are two or more of the corresponding elements. The term “multiplicity” is intended to mean that there are three or more of the corresponding elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A support apparatus for supporting at least a portion of a patient's limb during a procedure, said apparatus comprising:
a support having a surface for receiving at least a portion of the patient's limb thereon to support the limb during the procedure; and
a sensor mounted on the support such that the sensor is at least partially attached to the support without an adhesive, said sensor being adapted to communicate with the patient's limb for measuring a physiological parameter of the patient on the limb when the limb is supported by the support.

2. Apparatus in accordance with claim 1 wherein the surface has a contour generally corresponding to a portion of an arm of the patient's limb.

3. Apparatus in accordance with claim 1 wherein the surface has a contour generally corresponding to a portion of a hand of the patient's limb.

4. Apparatus in accordance with claim 1 wherein the sensor is mounted on the support generally adjacent the surface so the sensor is adapted to communicate with a portion of the patient's limb in contact with the surface during the procedure for measuring the physiological parameter of the patient on the limb.

5. Apparatus in accordance with claim 1 further comprising a restraint member on the support for restraining movement of at least a portion of the patient's limb during the procedure.

6. Apparatus in accordance with claim 1 wherein the sensor is selected from a group of sensors consisting of a temperature sensor, a pulse sensor, a blood pressure sensor, an electrocardiograph, and an oxygen saturation sensor.

7. A support apparatus for restraining movement of at least a portion of a patient's limb during a procedure, said apparatus comprising:
a support for receiving at least a portion of the patient's limb thereon to support the limb during the procedure; and
a restraint mounted on the support for engaging the limb to restrain movement of the limb, said restraint including a sensor adapted to communicate with the patient's limb for measuring a physiological parameter of the patient on the limb when the limb is received by the restraint.

8. Apparatus in accordance with claim 7 wherein the sensor is selected from a group of sensors consisting of a temperature sensor, a pulse sensor, a blood pressure sensor, an electrocardiograph, and an oxygen saturation sensor.

9. Apparatus in accordance with 7 wherein the restraint comprises a strap.

10. Apparatus in accordance with claim 9 wherein the strap is adapted to receive a portion of an arm of the patient's limb for restraining movement of the arm and for measuring the physiological parameter on the arm.

11. Apparatus in accordance with claim 9 wherein the strap is adapted to receive a portion of a hand of the patient's limb for restraining movement of the hand and for measuring the physiological parameter on the hand.

12. Apparatus in accordance with claim 7 wherein the restraint comprises a glove adapted to receive at least a portion of a hand of the patient's limb for restraining movement of the hand.

13. Apparatus in accordance with claim 12 wherein the glove comprises a first portion adapted to receive at least a portion of a first digit of the hand, and a second portion separate from the first portion and adapted to receive at least a portion of a second digit of the hand.

14. Apparatus in accordance with claim 12 wherein the glove includes a sleeve adapted to receive at least a portion of an arm of the patient's limb for restraining movement of the arm and for measuring the physiological parameter on the arm.

15. A support apparatus for restraining movement of at least a portion of a patient's limb during a procedure, said apparatus comprising:
a support for receiving at least a portion of the patient's limb thereon to support the limb during the procedure; and
a glove mounted on the support and adapted to receive at least a portion of a hand of the patient's limb, wherein said glove has a first portion adapted to receive at least a portion of a first digit of the hand, and a second portion separate from the first portion and adapted to receive at least a portion of a second digit of the hand.

16. Apparatus in accordance with claim 15 wherein a sleeve extends from the glove and is adapted to receive at least a portion of an arm of the patient's limb for restraining movement of the arm.

17. A glove assembly for measuring a physiological parameter of patient during a procedure, said assembly comprising:
a glove adapted to receive at least a portion a hand of the patient;
a sleeve extending from the glove and adapted to receive at least a portion of an arm of the patient, said sleeve adapted to extend beyond a wrist of the patient and along at least a portion of the arm when the arm is received by the sleeve; and
a sensor mounted on at least one of the glove and the sleeve, said sensor being adapted to communicate with the patient for measuring the physiological parameter when the arm is received by the sleeve.

18. A glove assembly in accordance with claim 17 wherein the sensor is selected from a group of sensors consisting of a temperature sensor, a pulse sensor, a blood pressure sensor, an electrocardiograph, and an oxygen saturation sensor.

19. A glove assembly in accordance with claim 17 wherein the glove has a first portion adapted to receive at least a portion of a first digit of the hand, and a second portion separate from the first portion and adapted to receive at least a portion of a second digit of the hand.

20. A glove assembly in accordance with claim 17 wherein the sensor is mounted on the sleeve and adapted to communicate with the patient's arm for measuring the physiological parameter on the arm.

21. A support apparatus for supporting at least a portion of a patient's limb during a procedure, said apparatus comprising:
a support having a surface for receiving at least a portion of the patient's limb thereon to support the limb during the procedure; and
a sensor mounted on the support and including a measuring region facing away from the support surface for engaging the patient's limb when received by the support for measuring a physiological parameter of the patient.

22. Apparatus in accordance with claim 21 wherein the surface has a contour generally corresponding to a portion of an arm of the patient's limb.

23. Apparatus in accordance with claim 21 wherein the surface has a contour generally corresponding to a portion of a hand of the patient's limb.

24. Apparatus in accordance with claim 21 further comprising a restraint on the support for restraining movement of at least a portion of the patient's limb during the procedure.

25. Apparatus in accordance with claim 21 wherein the sensor is selected from a group of sensors consisting of a temperature sensor, a pulse sensor, a blood pressure sensor, an electrocardiograph, and an oxygen saturation sensor.
